(19) United States
(12) Patent Application Publication
(10) Pub. No.: US 2023/0159690 A1
(43) Pub. Date: May 25, 2023

(54) SYNTHESIS AND APPLICATIONS OF POLYMERS OF PENTADECYL CAPROLACTONE

(71) Applicant: Regents of the University of Minnesota, Minneapolis, MN (US)

(72) Inventors: Christopher J. Ellison, Eden Prairie, MN (US); Marc A. Hillmyer, Minneapolis, MN (US); Hee Joong Kim, Minneapolis, MN (US)

(21) Appl. No.: 17/917,848

(22) PCT Filed: Apr. 7, 2021
(86) PCT No.: PCT/US2021/026230

§ 371 Date: Oct. 7, 2022

Related U.S. Application Data
(60) Provisional application No. 63/006,470, filed on Apr. 7, 2020.

(51) Int. Cl. C08F 293/00; C09J 167/04

(52) U.S. Cl. CPC C08F 293/00; C09J 167/04

(57) ABSTRACT

Polymers, including homopolymers, copolymers, block copolymers, comprising poly(pentadecyl caprolactone), methods of making the polymers, and pressure sensitive adhesives including the polymers.

### CROSS-REFERENCE TO RELATED APPLICATIONS

This application claims the benefit of U.S. Patent Application No. 63/006,470, entitled “SYNTHESIS AND APPLICATIONS OF POLYMERS OF PENTADECYL CAPROLACTONE” and filed on Apr. 7, 2020.

### STATEMENT OF GOVERNMENT INTEREST

This invention was made with government support under CHE-1901635 awarded by National Science Foundation. The government has certain rights in the invention.

### TECHNICAL FIELD

This invention relates to synthesis of homopolymers and copolymers of pentadecyl caprolactone and compositions (e.g., pressure sensitive adhesives) including these polymers.

### BACKGROUND

Pressure sensitive adhesives (PSAs) are widely used in applications such as sticky notes, labels, tape, and stamps due to their facile adhesion to substrates with a minimal applied force. Most commercial PSA materials are derived from nonrenewable resources (e.g., fossil resources). Sustainable PSA materials synthesized from renewable resources with (e.g., acrylic acid) may have limited adhesion or degradability. Generally, PSAs are associated with “residual adhesive” problems during the paper recycling process in pulping facilities. It has been a challenge to design new PSA materials from sustainable resources possessing competitive adhesion properties and degradability.

### SUMMARY

A first general aspect includes synthesizing a polymer includes polymerizing pentadecyl caprolactone to yield poly(pentadecyl caprolactone).

Implementations of the first general aspect may include one or more of the following features.

In some implementations, polymerizing pentadecyl caprolactone comprises polymerizing pentadecyl caprolactone with one or more additional monomers to yield a copolymer comprising poly(pentadecyl caprolactone). The one or more additional monomers can include one or more additional lactone monomers.

In some cases, the copolymer is a block copolymer comprising poly(pentadecyl caprolactone). The block copolymer can include a first block type and a second block type, wherein the first block type and the second block type are different. In certain cases, the one or more additional monomers comprise a lactide. When the one or more additional monomers comprise a lactide, the first block type comprises poly(lactic acid) and the second block type comprises poly(pentadecyl caprolactone). The poly(lactic acid) can comprise or consist essentially of poly(L-lactic acid).

In some cases, the block copolymer further comprises a third block type. The third block type can be the same as the first block type, the same as the second block type, or different than the first block type and the second block type. The block copolymer can be an ABA triblock copolymer, where A comprises the poly(lactic acid) and B comprises the poly(pentadecyl caprolactone).

In some implementations, the pentadecyl caprolactone is an isomeric mixture. The isomeric mixture can include 3-pentadecyl caprolactone and 5-pentadecyl caprolactone. Some implementations of the first general aspect include reducing cardanol to yield the isomeric mixture. Some implementations of the first general aspect include processing cashew nut shell liquid to yield the cardanol. In some cases, when the pentadecyl caprolactone is an isomeric mixture, the isomeric mixture comprises about 25 wt % 3-pentadecyl caprolactone and about 75 wt % 5-pentadecyl caprolactone. In certain cases, reducing the cardanol comprises catalyzed hydrogenation under a pressure of at least about 35 bar for at least about 1 hour.

Some implementations of the first general aspect include combining the polymer (e.g., the copolymer or block copolymer) with a tackifier to yield a pressure sensitive adhesive. In some cases, the poly(pentadecyl caprolactone) is miscible with the tackifier.

A second general aspect includes a polymer comprising poly(pentadecyl caprolactone).

Implementations of the second general aspect may include one or more of the following features.

In some implementations, the polymer is a copolymer. In some implementations, the copolymer is a block copolymer. The block copolymer can include a first block type and a second block type, with the first block type and the second block type being different. In some implementations, the block copolymer further comprises a third block type. The third block type can be the same as the first block type, the same as the second block type, or different than the first block type and the second block type. In some implementations, the first block type comprises poly(lactic acid).

In some implementations, the polymer is an ABA triblock copolymer. In one example, the ABA triblock copolymer is represented by the following chemical structure:

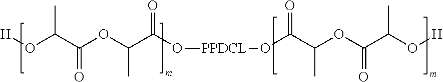

wherein each m is independently an integer, and PPDCL is represented by the following chemical structure:

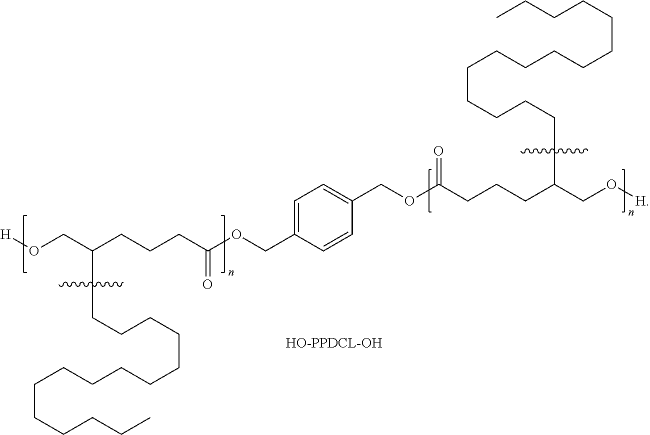

In some implementations, the first block type comprises, consists essentially of, or consists of poly(L-lactic acid), and the ABA triblock copolymer is represented by the following chemical structure:

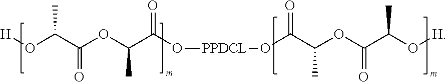

In some implementations, the block copolymer degrades to carbon-containing molecules, each carbon-containing molecule having a number of carbon atoms in a range from 20 to 200.

In some implementations in which the polymer is a block copolymer, the first block type and the second block type are immiscible. In certain cases in which the polymer is a block copolymer, the first block type comprises a polymer having a glass transition temperature exceeding that of PPDCL.

In some implementations, the block copolymer is represented by one of the following chemical structures:

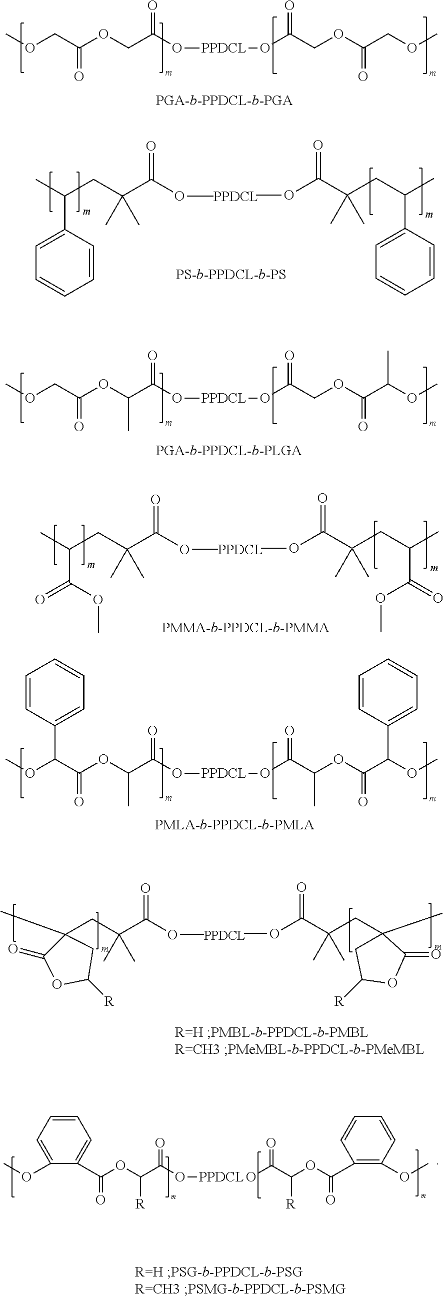

In some implementations, the polymer is a block copolymer, and a glass transition temperature of the first block type is greater than 25° C. In some examples, the first block type comprises poly(glycolic acid) (PGA), poly(lactic acid-co-glycolic acid) (PLGA), poly(mandelic acid-co-lactic acid) (PMLA), poly(salicylic glycolid) (PSG), poly(salicylic methyl glycolide) (PSMG), poly(styrene) (PS), poly(methyl methacrylate) (PMMA), cpoly(α-methylene-γ-butyrolactone) (PMBL), or poly(γ-methyl-α-methylene-γ-butyrolactone) (PMeMBL).

A third general aspect includes a pressure sensitive adhesive comprising the polymer of the second general aspect.

Implementations of the third general aspect may include one or more of the following features.

In some implementations, the pressure sensitive adhesive further comprises a tackifier. In some implementations, the polymer is a block copolymer and one of the blocks of the block copolymer is miscible with the tackifier. In certain implementations, the pressure sensitive adhesive comprises about 5 wt % to about 80 wt % or about 20 wt % to about 80 wt % of the tackifier.

In one example, an aliphatic polyester with a long alkyl substituent, poly(pentadecyl caprolactone) (PPDCL), is synthesized from lactones (e.g., lactones derived from cashew nut shell liquid (CNSL)) by ring opening transesterification polymerization (ROTEP) in a controlled fashion. The PPDCL can be used as a rubbery central block in symmetric triblock copolymers (e.g., ABA triblock copolymers) with glassy poly(lactide) end blocks. FIG. 1 depicts an example of a synthesis of a triblock copolymer from PPDCL (e.g., from cashew nut shell) and lactide (e.g., from corn). The ABA triblock copolymers can be blended with a renewable tackifier to yield sustainable pressure sensitive (PSA) materials. The tackifier increases the adhesion by diluting entanglements in the central block, thus lowering the modulus. The resultant PSAs show competitive adhesion properties compared to commodity adhesives. In addition, the ABA triblock copolymers hydrolytically degrade at 50° C. under acidic conditions.

In one example, poly(pentadecyl caprolactone) (PPDCL) is synthesized from cashew nut shell liquid derived lactones by ring opening transesterification polymerization. Synthesis of symmetric triblock copolymers including a PPDCL central block and poly(lactide) end blocks is described. The ABA triblock copolymers are blended with a tackifier to produce pressure sensitive adhesive (PSA) materials. The adhesion properties can be tuned by altering the polymer/tackifier. In one example, the resultant PSA has adhesion properties comparable to commercial adhesives of the permanent adhesive level. In addition, the PPDCL homopolymers and triblock copolymers containing PPDCL central block hydrolytically degrade at 50° C. under acidic conditions. These adhesives can be useful for removable PSA applications, including indoor applications: they strongly adhere to a substrate and can be detached easily and cleanly on demand. Suitable applications include sticky notes, box closing tape, labels, electrical tape, office tape, masking tape, duct tape, and other adhesive tape. Additional applications include heat-activated adhesives (e.g., adhesives activated by body temperature).

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1 depicts synthesis of a triblock copolymer from cashew nut shell and corn.

FIG. 2 shows a synthetic route for PDCL from cashew nut shells.

FIG. 3 shows synthetic routes for PPDCL, LPL, and cLPL.

FIGS. 4A-4C related to bulk polymerization of PDCL at 110° C., [PDCL]₀=2.91 M, [Sn(Oct)₂]₀=0.015 M. FIG. 4A shows conversion of PDCL as a function of reaction time ([BDM]₀=0.015 M). The inset shows linear fit of kinetic data up to 9 h ([M]_{eq}=0.23 M). FIG. 4B shows a linear relationship between conversion and molar mass ([BDM]0=0.015 M). FIG. 4C shows molar mass control of PPDCL. Each polymerization was performed to the equilibrium conversion and the dashed lines indicate the theoretical molar mass at the equilibrium conversion. Conversion of PDCL and molar mass was determined by ¹H NMR spectroscopy. Dispersity (Ð) was determined by MALS-SEC (THF, 25° C.).

FIG. 5A shows DSC data (second heating, 10° C. min⁻¹). FIG. 5B shows SAXS data (at 40° C., vertically shifted for clarity). FIG. 5C shows storage moduli (cooling, 10° C. min⁻¹, 1 rad s⁻¹) of the synthesized polymers.

FIG. 6 shows DSC data (second heating, 10° C. min⁻¹) of LPL (114 k, 0.13) with different tackifier composition. The vertical dashed line indicates room temperature (23° C.).

FIGS. 7A-7C show adhesion properties of LPLs, cLPL, and commercial adhesives. FIG. 7A shows peel adhesion. FIG. 7B shows loop tack adhesion. FIG. 7C shows shear resistance. Open and filled symbols indicate cohesive and adhesive failure, respectively. Horizontal lines indicate the adhesion properties of commercial adhesives. All of the peel and loop tack adhesion tests were performed at the rate of 305 mm min⁻¹, otherwise noted. #=measured at a peel rate of 31 mm min-1. *=No failure for 6000 min, and then the experiment was manually terminated.

FIG. 8 shows relative remaining molar mass (M_{n,MALS-SEC}) of PPDCL and LPLs under hydrolytic degradation conditions at 50° C. in 0.1 M HCl(aq)/THF mixture solution (1/1 vol.).

FIG. 9 depicts copolymerization of pentadecyl caprolactone and methyl caprolactone.

### DETAILED DESCRIPTION

This disclosure describes synthesis and applications of poly(pentadecyl caprolactone) (PPDCL) polymers, including homopolymers, copolymers, and block copolymers. PPDCL is synthesized by polymerizing pentadecyl caprolactone (PDCL) under conditions described herein. In some cases, PDCL is polymerized with one or more monomers (in addition to PDCL) to yield a copolymer comprising PPDCL. In one example, the one or more additional monomers include one or more additional lactone monomers. The copolymer is a block copolymer comprising PPDCL. The block copolymer can include a first block type and a second block type. The first block type and the second block type are different (e.g., an AB diblock copolymer). In another example, the one or more additional monomers comprise a lactide. When the one or more additional monomers comprise a lactide, the first block type comprises poly(lactic acid) (PLA) and the second block type comprises PPDCL. The PLA can comprise, consist essentially of, or consist of poly(L-lactic acid).

In some cases, the block copolymer includes a third block type. The third block type can be the same as the first block type, the same as the second block type, or different than the first block type and the second block type. In one example, the block copolymer is an ABA triblock copolymer, where A comprises PLA and B comprises PPDCL.

The PDCL can be an isomeric mixture. In one example, the isomeric mixture includes 3-pentadecyl caprolactone and 5-pentadecyl caprolactone. In some cases, cardanol is reduced to yield the isomeric mixture. In one example, the cardanol is synthesized by processing cashew nut shell liquid. When the pentadecyl caprolactone is an isomeric mixture, the isomeric mixture comprises about 25 wt % 3-pentadecyl caprolactone and about 75 wt % 5-pentadecyl caprolactone. In one example, reducing the cardanol comprises catalyzed hydrogenation under a pressure of at least about 35 bar for at least about 1 hour.

The PPDCL polymer (e.g., the copolymer or block copolymer) can be combined with a tackifier as described herein to yield a pressure sensitive adhesive. In some cases, the polymer (e.g., the copolymer or block copolymer) is miscible with the tackifier.

This disclosure also describes a polymer comprising PPDCL and applications thereof. The polymer can be a copolymer or a block copolymer. The block copolymer can include a first block type and a second block type, with the first block type and the second block type being different (e.g., an AB block copolymer). The block copolymer can include a third block type. The third block type can be the same as the first block type, the same as the second block type, or different than the first block type and the second block type. In some implementations, the first block type comprises PLA.

The copolymer can be an ABA triblock copolymer. In one example, the ABA triblock copolymer is represented by the following chemical structure:

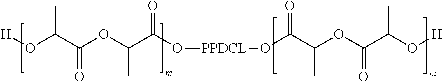

wherein each m is independently an integer, and PPDCL is represented by the following chemical structure:

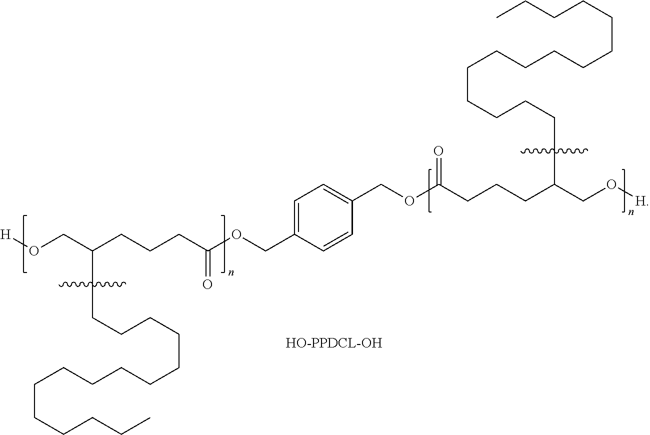

In some examples, the first block type comprises, consists essentially of, or consists of poly(L-lactic acid), and the ABA triblock copolymer is represented by the following chemical structure:

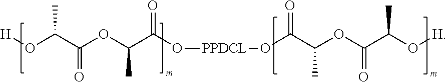

The block copolymer can be degraded to carbon-containing molecules, each carbon-containing molecule having a number of carbon atoms in a range from 20 to 200.

When the polymer is a block copolymer, the first block type and the second block type can be immiscible. When the polymer is a block copolymer, the first block type can include a polymer having a glass transition temperature exceeding that of PPDCL. In some cases, a glass transition temperature of the first block type is greater than 25° C.

Examples of block copolymers are depicted below.

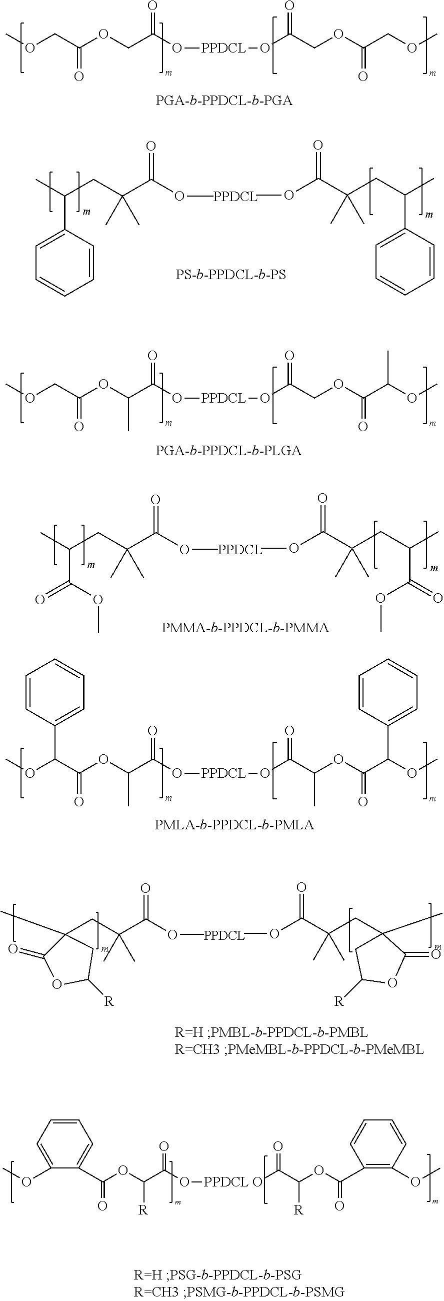

In some examples, the first block type comprises poly(glycolic acid) (PGA), poly(lactic acid-co-glycolic acid) (PLGA), poly(mandelic acid-co-lactic acid) (PMLA), poly(salicylic glycolid) (PSG), poly(salicylic methyl glycolide) (PSMG), poly(styrene) (PS), poly(methyl methacrylate) (PMMA), cpoly(α-methylene-γ-butyrolactone) (PMBL), or poly(γ-methyl-α-methylene-γ-butyrolactone) (PMeMBL).

This disclosure also describes pressure sensitive adhesives including a polymer comprising PPDCL. In some cases, the pressure sensitive adhesive includes a tackifier as described herein. The tackifier can include one or more of hydrocarbon resins, rosin acids, rosin esters, and terpene resins. The polymer can be a block copolymer. When the polymer is a block copolymer, one of the block types of the block copolymer can be miscible with the tackifier. In some examples, the pressure sensitive adhesive includes about 5 wt % to about 80 wt % or about 20 wt % to about 80 wt % of the tackifier.

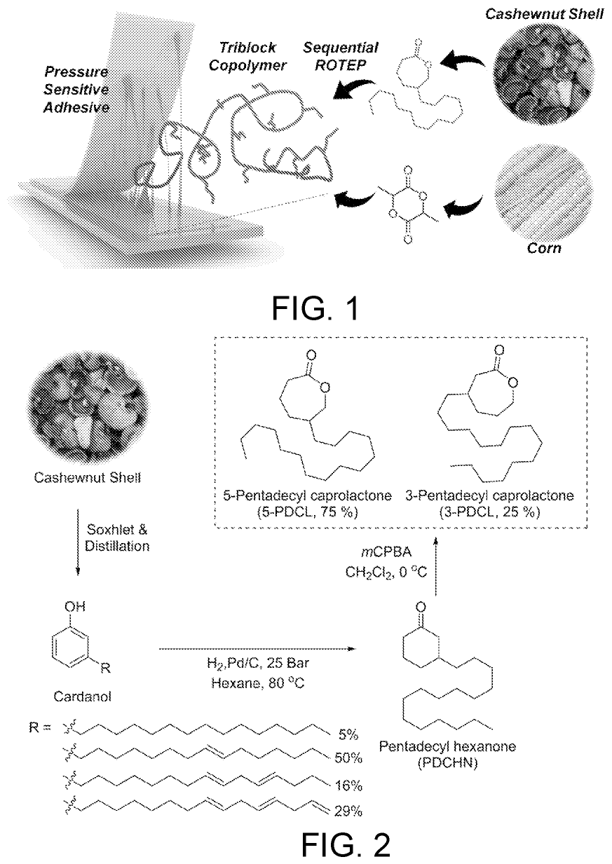

In one example, an aliphatic polyester with a long alkyl substituent, poly(pentadecyl caprolactone) (PPDCL), is synthesized from lactones (e.g., lactones derived from cashew nut shell liquid (CNSL)) by ring opening transesterification polymerization (ROTEP) in a controlled fashion. The PPDCL can be used as a rubbery central block in symmetric triblock copolymers (e.g., ABA triblock copolymers) with glassy poly(lactide) end blocks. FIG. 1 depicts an example of a synthesis of a triblock copolymer from PPDCL (e.g., from cashew nut shell) and lactide (e.g., from corn). The ABA triblock copolymers can be blended with a renewable tackifier to yield sustainable pressure sensitive (PSA) materials. The tackifier increases the adhesion by diluting entanglements in the central block, thus lowering the modulus. The resultant PSAs show competitive adhesion properties compared to commodity adhesives. In addition, the ABA triblock copolymers hydrolytically degrade at 50° C. under acidic conditions.

Monomer Synthesis. Cardanol, an ingredient in CNSLs, was used as the starting material to synthesize pentadecyl caprolactone (PDCL). As depicted in FIG. 2, cardanol is reduced by Pd-catalyzed hydrogenation, yielding a mixture of 3-pentadecyl cyclohexanol and 3-pentadecyl cyclohexanone (PDCHN). A pressure of at least about 35 bar and a reaction time of at least about 6 h promotes high yields of PDCHN (˜70%). The PDCHN is converted to the caprolactone form (PDCL) by Baeyer-Villiger oxidation with meta-chloroperoxybenzoic acid (mCBPA), yielding a regioisomeric mixture (PDCL) containing 75% 5-PDCL and 25% 3-PDCL.

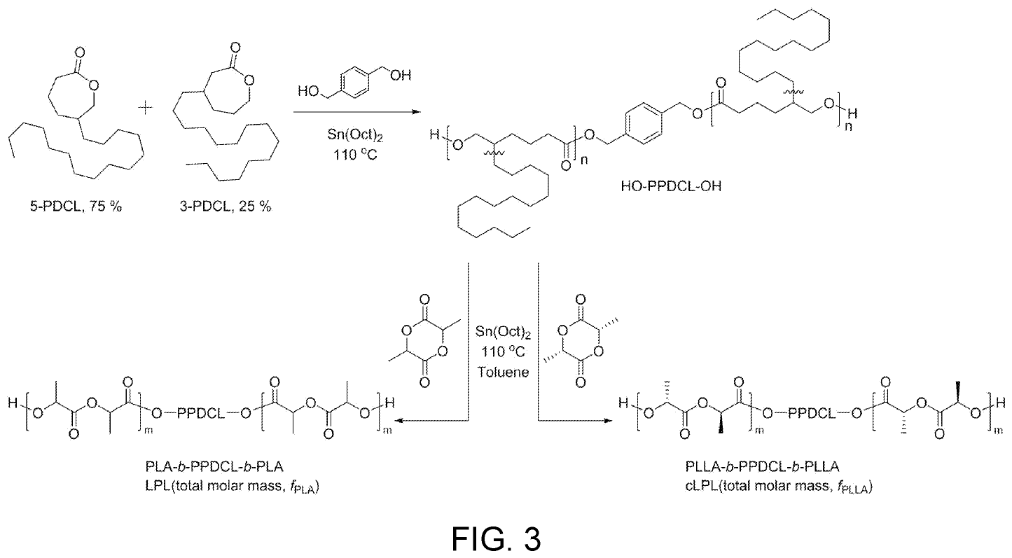

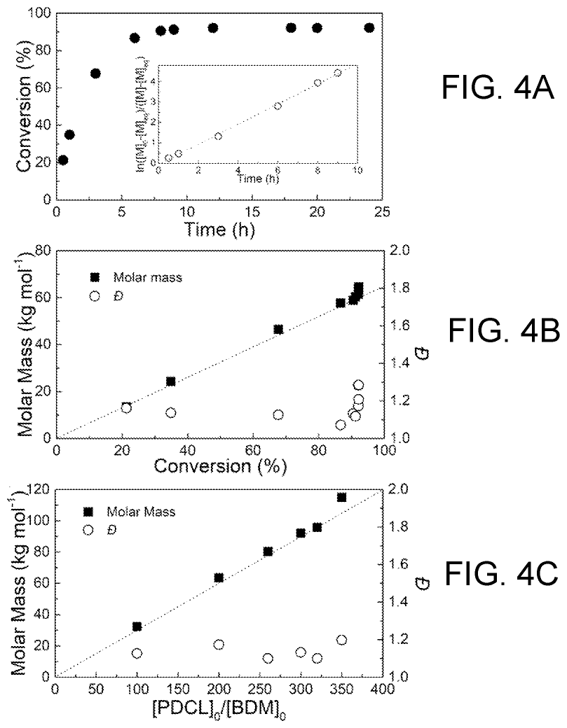

Bulk Polymerization. As depicted in FIG. 3, bulk polymerization of PDCL can be carried out at 110° C. using Sn(Oct)₂ as a catalyst and 1,4-benzenedimethanol (BDM) as an initiator. The bulk polymerization can be monitored over time by analyzing aliquots using ¹H NMR spectroscopy and size exclusion chromatography equipped with multiangle light scattering (MALS-SEC) at 25° C. using tetrahydrofuran (THF) eluent. As shown in FIG. 4A, the conversion of PDCL reached 87% after 6 h, plateauing at about 92% after 9 h. The kinetic plot in the inset indicates the polymerization is first-order in monomer, and the pseudo first-order rate constant was 1.4×10⁻⁴ s⁻¹ at [Sn(Oct)₂]₀=0.015 M. By assuming first order dependence on catalyst concentration, the second-order rate constant was determined to be 9.1×10⁻³M⁻¹s⁻¹. As shown in FIG. 4B, a linear relationship between the conversion and molar mass was observed, suggesting a controlled polymerization process. The increase in dispersity that occurs at 92% conversion is believed to be due to intermolecular transesterification and/or other phenomena. As shown in FIG. 4C, molar mass of PPDCL increased linearly with increasing [PDCL]₀/[BDM]₀ at fixed conversion, resulting in molar mass up to 115 kg mot′ (M_{n,NMR}), which is additional evidence for a controlled polymerization. 5-PDCL was slightly more reactive than 3-PDCL during the polymerization.

The thermodynamics of the ROTEP of PDCL were investigated by measuring the equilibrium monomer concentration at different polymerization temperatures. By defining standard monomer concentration ([M]ₛₛ) as 1.0 M, the thermodynamic parameters were calculated using eq. 1.

$${ln [ M ]}_{eq} = \frac{\text{?}}{RT} - \frac{\text{?}}{R} \qquad ( eq . 1 )$$

$$\text{?} \text{indicates text missing or illegible when filed}$$

From a linear plot of ln[M]_{eq} versus T⁻¹, the enthalpy and entropy of PDCL polymerization were determined to be ΔH_{P}^{o}=−16.9±2.1 kJ mol-1 and ΔS_{P}^{o}=−31.2±5.3 J mol⁻1K⁻¹, respectively.

Synthesis of ABA Triblock Copolymers. PLA-b-PPDCL-b-PLA triblock copolymers (LPLs) are synthesized by the ROTEP of lactide using high molar mass (≈100 kg mol⁻¹) hydroxytelechelic PPDCL as a macro-initiator (see FIG. 2). Typical styrenic triblock copolymers for PSA applications have small PS volume fractions. Therefore, small volume fractions of PLA end blocks (f_{PLA}=0.06 and 0.13) were targeted (Table 1). The LPL with a high PLA composition (f_{PLA}=0.50), LPL(82 k, 0.50), was also synthesized for clearer observation of the T_{g,PLA}. ¹³C NMR spectra revealed no evidence of transesterification between PPDCL and PLA blocks, indicating that the block copolymers have the desired ABA architecture In addition, PLLA-b-PPDCL-b-PLLA, cLPL(107 k, 0.14), containing similar PLLA fractions was synthesized by the ROTEP of L-lactide using hydroxytelechelic PPDCL macro-initiator, allowing investigation of the effect of crystallinity in the hard segments on the adhesion properties.

TABLE 1
Molecular characterization data for the synthesized polymers

| Polymer | M_{n,PPDCL}^{a} | M_{n,PLA}^{a} | M_{n,total}^{a} | M_{n,MALS-SEC}^{a} | M_{w,MALS-SEC}^{b} | Ð^{b} | ƒ_{PLA}^{c} |
|---|---|---|---|---|---|---|---|
| PPDCL (92 k) | 92 | 0 | 92 | 71 | 89 | 1.3 | 0 |
| LPL (125 k, 0.06) | 115 | 10 | 125 | 102 | 124 | 1.2 | 0.06 |
| LPL (114 k, 0.13) | 96 | 18 | 114 | 107 | 129 | 1.2 | 0.13 |
| LPL (82 k, 0.50) | 35 | 47 | 82 | 67 | 86 | 1.3 | 0.50 |
| cLPL (l 07 k, 0.14) | 88 | 19 | 107 | 96 | 114 | 1.2 | 0.14 |

^{a}Determined by ¹H NMR spectroscopy (kg mol⁻¹).
^{b}Determined by MALS-SEC (THF, 25° C.) (molar mass unit, kg mol⁻¹).
^{c}Calculated using ρ_{PPDCL} = 0.943 g cm⁻³ at 0° C. and ρ_{PLA} = 1.25 g cm⁻³ at 25° C.

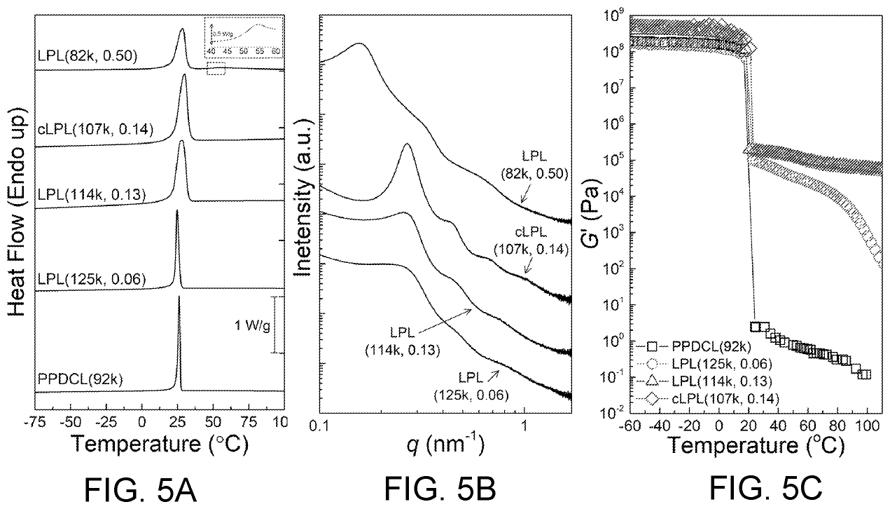

Polymer Properties. Thermal, morphological, and rheological properties of the polymers were evaluated using differential scanning calorimetry (DSC), small-angle X-ray scattering (SAXS), and dynamic mechanical analysis (DMA), respectively. As seen in FIG. 5A, PPDCL(92 k) showed a clear melting peak at 27° C. which can be attributed to the long alkyl (C15) side chains in PPDCL. This T_{m,PPDCL} is comparable to the Tₘ of poly(n-alkyl acrylates) (n=14-18). No clear transition could be assigned to T_{g},PPDCL, due in part to its large melting peak. Instead, T_{g},PPDCL was determined by DMA, which displayed a broad tan δ peak at −60° C. This is comparable to T_{g,PγMC}L (−60° C.). Block copolymers containing small PLA fractions showed T_{m,PPDCL}≈27° C., whereas T_{g,PLA} was not clear likely because of the small PLA content. Alternatively, the T_{g,PLA} of LPL(125 k, 0.06), LPL(114 k, 0.13), and cLPL(107 k, 0.14) were observed at 45° C., 55° C., and 59° C., respectively, by DMA. Additionally, LPL(82 k, 0.50) exhibited a distinct T_{g,PLA}≈48° C. and T_{m,PPDCL}≈27° C. by DSC. The T_{m,PPDCL} of block copolymers are close to that of PPDCL homopolymer regardless of the volume fraction of PLA, suggesting that the two blocky components are immiscible. As shown in FIG. 5B, the microphase-separations were corroborated by small angle X-ray scattering profiles of the block copolymers. All of the block copolymers showed prominent principle scattering peaks, suggesting they have a microphase separated structure. Although the morphologies of block copolymers containing low PLA content (f_{PLA}<0.15) are difficult to fully assign due to the broadness and weak intensity of the higher-order reflections, it is believed that they exhibit spherical morphologies given the peak position ratio. Similar scattering patterns of triblock copolymers have are believed to be a disorganized spherical morphology with the absence of long-range order. The LPL(82 k, 0.50) sample exhibits lamellae, which is consistent with the expectation for a symmetric block copolymer (f_{PLA}≈0.5).

The dynamic elastic modulus is a parameter governing the wetting of a substrate by PSA. According to the Dahlquist criterion, the storage modulus (G′) of PSA at the operating temperature should be less than 3×105 Pa in order to have adequate wetting. As shown in FIG. 5C, all of the block copolymers showed a significant decrease in G′ in the vicinity of T_{m,PPDCL}≈21° C., then showed plateau moduli. The plateau moduli of LPL(125 k, 0.06), LPL(114 k, 0.13), and cLPL(107 k, 0.14) (9.4×10⁴, 1.9×10⁵, and 1.9×10⁵ Pa at 25° C., respectively) fall within the Dahlquist criterion. LPL(125 k, 0.06) showed a lower modulus than those of LPL(114 k, 0.13) and cLPL(107 k, 0.14) in the entire temperature range of interest due to the lower PLA content. The Mₑ of PPDCL was estimated by employing the Guth-Smallwood equation. Based on the plateau moduli of LPL(125 k, 0.06) and LPL(114 k, 0.13) at 25° C., M_{e,PPDCL}≈20-28 kg mol⁻¹ was approximated. The M_{e,PPDCL} is comparable to the Mₑ of poly(n-butyl acrylate) (≈28 kg mol⁻¹) but larger than the other central blocks used in degradable ABA triblock copolymers for PSAs. Therefore, LPL-based PSAs are expected to exhibit strong adhesion to substrates.

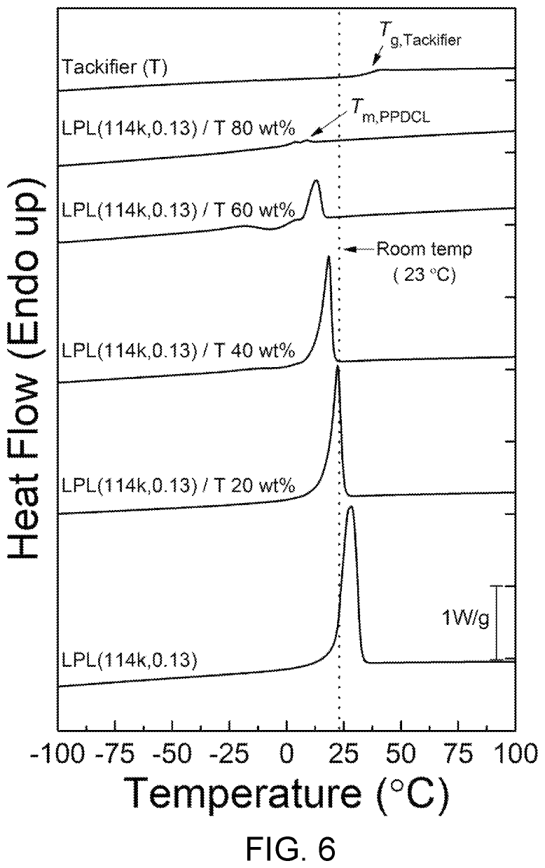

Adhesion Properties. PSA formulations were prepared by solution mixing LPLs with a rosin ester tackifier (Sylvalite 2E 80HP). By adding the tackifier, additional tuning of adhesion and cost reduction are possible. Increasing the tackifier content leads to a decrease in T_{m,PPDCL} as well as the decrease in crystallinity of the PPDCL (see FIG. 6, suggesting PPDCL and tackifier are miscible). In contrast, T_{g,PLA} measured by DMA was almost constant (55° C.) when 20 wt % tackifier is blended, indicating that PLA is not miscible with the tackifier. Therefore, the tackifier is miscible with only the less polar PPDCL central block.

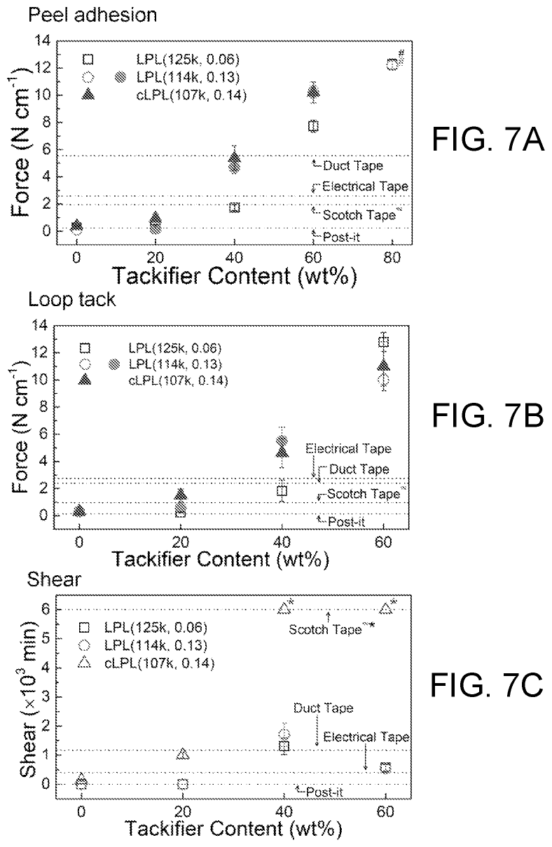

The adhesion properties of the resultant PSAs such as peel adhesion, loop tack, and shear strength were evaluated. Results are shown in FIGS. 7A-7C. The neat block copolymers with relatively high M_{e,PPDCL} exhibited a low level of adhesion (peel adhesion 0.19 N cm⁻¹, loop tack adhesion ≈0.15 N cm⁻¹, as shown in FIGS. 7A and 7B), which is comparable to a POST-IT note. The peel and tack adhesion were not improved even after the incorporation of 20 wt % tackifier. This is likely because the PPDCL block cannot readily flow and adhere to the surface since T_{m,PPDCL} is above the room (operating) temperature (≈23° C.) (see FIG. 6). For neat block copolymers or PSAs containing 20 wt % tackifier, the peel adhesion (measured at room temperature) increased up to 0.7 N cm⁻¹ when the substrate surface was heated to 38° C. for 15 s to improve the flow during adhesion. This suggests that these adhesives would also be useful as heat-activated adhesives, and even could be activated by body temperature. Increasing tackifier content beyond 20 wt % led to a significant improvement in peel and loop tack adhesion due to decreases in T_{m,PPDCL} below the operating temperature, enabling adequate flow upon light pressure. For example, a peel force of 4.71±0.46 N cm⁻¹ and a loop tack force of 5.51±1.00 N cm⁻¹ were obtained for LPL(114 k, 0.13) with 40 wt % tackifier content, which are comparable to commercial adhesives (e.g., SCOTCH tape, electrical tape). Increasing the tackifier content further to 60 wt % achieves excellent peel and loop tack adhesion, which are superior to these commercial adhesives. Given that the Tₘ of the PSA is still higher than 0° C., PPDCL can partially crystallize at lower temperatures. When an adhered adhesive surface was cooled down by an ice cube for 20 s, the peel force decreased accompanied by adhesive failure (i.e., no adhesive residue on a substrate after failure). This suggests that the adhesive can strongly adhere to a substrate and can be detached easily and cleanly on demand, which could be useful for indoor applications.

Overall, LPL(114 k, 0.13) and cLPL(107 k, 0.14) exhibited similar or stronger peel/loop tack adhesion than LPL(125 k, 0.06) at most tackifier compositions due to the higher PLA content. One exception is that LPL(125 k, 0.06) exhibited stronger loop tack adhesion than the others when the tackifier content was 60 wt %, presumably because its modulus was optimized to be tack. Adhesive failures were observed for cLPLs in the entire range of tackifier compositions, suggesting that they would be useful for removable PSA application. This is likely because PLLA block contributed to change the ductility of the formed fibrils upon peeling, thereby they detached from the substrate instead of snapping within themselves although the mechanisms for cohesive/adhesive failures have not been fully established yet due to its complexity (e.g., cavitation, elastic hardening, speed). Surprisingly, transparent PSAs with 80 wt % tackifier were prepared without phase separation between the LPLs and the tackifier. However, stick slip failures were observed for those samples when the peel test was performed at a peel rate of 305 mm min⁻¹ possibly due to the partial coverage of the substrate by free tackifier. Reliable peel adhesion data were obtained at a slow peel rate of 31 mm min⁻¹. Note that common tackifier compositions for PSAs are lower than 60 wt % for reasons including: either an increase in T_{g} to near/above use temperature or phase separation between the polymer and the tackifier are observed. For example, a drop in peel adhesion was observed when the PLA-PM-PLA was blended with 60 wt % tackifier. The LPL system reported here can apparently tolerate more tackifier than other ABA triblock copolymer systems (e.g., PLA-b-PM-b-PLA and PLA-b-PβMδVL-b-PLA), possibly due to the nonpolar nature of PPDCL.

To examine the interfacial resistance of PSA to flow under a constant load, shear tests were carried out by suspending a 500 g weight on the adhesive film (contact area: 1.27 cm×1.27 cm) adhered to the substrate. Particularly, LPLs with 40 or 60 wt % tackifier failed after ˜500 and 1700 min, respectively, which is comparable to electrical tape, but not as good as the Scotch Tape™ (see FIG. 7C). This is due to the weak resistance to flow of LPLs, which results from the low modulus of PPDCL and weak physical crosslinking of PLA hard segments. While, cLPL(107 k, 0.14) with 40 or 60 wt % tackifier showed no shear failure up to 6000 min, which is comparable to the SCOTCH Tape, likely because the strong physical crosslinking of crystalline domains in the PLLA hard segments provide strong resistance to creep. To summarize the adhesion of LPL-based PSAs, the adhesion as well as the failure mode can be tuned over a wide range (from POST-IT Note to stronger than SCOTCH Tape) by controlling the PLA length, crystallinity of PLA, and/or tackifier composition.

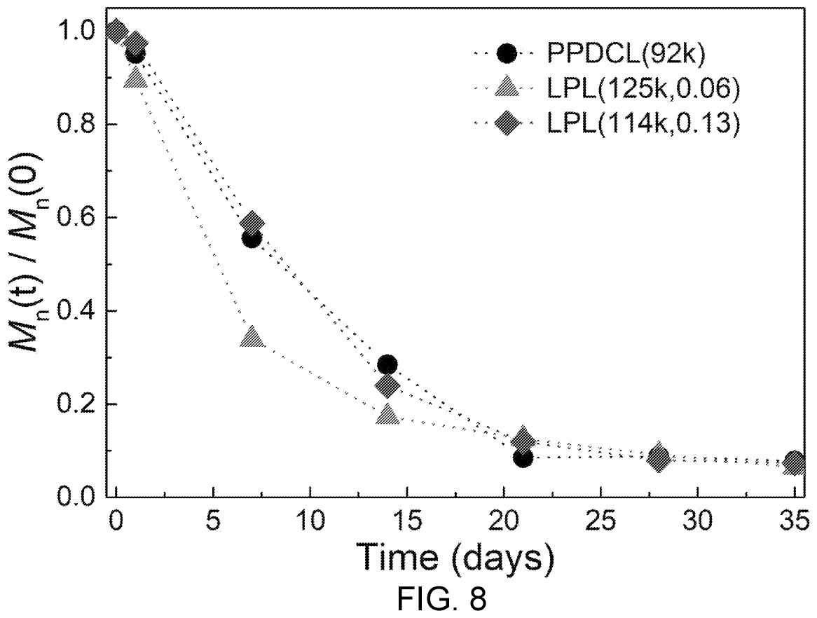

Hydrolytic Degradation. The hydrolytic degradation of PPDCL and LPLs at 50° C. in 0.1 M HCl/THF or 0.1M NaOH/THF mixture solutions (1/1 vol.) was monitored by MALS-SEC. When polymers were exposed to acidic conditions, all polymers exhibited more than 90% loss of molar mass (M_{n,MALS-SEC}) after 28 days compared to the original value (FIG. 8), suggesting that PPDCL degrades by acid-catalyzed hydrolysis as for poly(caprolactone) under similar conditions. This result demonstrates that the LPL-based PSA would be particularly useful for solving the “adhesive residue” problem in pulping facilities. The degradation trends for all the polymers are almost identical because they all contain a large amount of PPDCL block. The small differences in degradation rates are likely due to the small differences in hydrophilicity, original molar mass, and PLA content. A decrease in molar mass was accompanied by the broadening of SEC traces, which is evidence of homogeneous and random chain scission. The degradation of PPDCL was slower when exposed to basic condition.

### EXAMPLES

### Synthesis of Poly(Pentadecyl Caprolactone), Block Copolymers, and Pressure Sensitive Adhesive

An aliphatic polyester (e.g., poly(pentadecyl caprolactone) with a long alkyl substituent was synthesized in a controlled manner from a cardanol based lactone. The poly(pentadecyl caprolactone (PPDCL) was used as a first block in a block copolymer with polylactide (PLA) as a second. This block copolymer (poly(lactide)-block-poly(pentadecyl caprolactone)-block-poly(lactide), or LPL) was demonstrated as a pressure sensitive adhesive (PSA) material, by either using a neat material or blending with a renewable tackifier. The resultant PSAs exhibited a wide range of adhesion properties. The unique and beneficial characteristics of LPL-based PSAs can be attributed at least in part to the long alkyl substituent in the PPDCL block. In addition, the LPLs degraded in 0.1 M HCl(aq)/THF mixture solution at 50° C. within a reasonable time frame. The properties of these polymeric materials can be further tuned by controlling molar mass, architecture, and composition.

Materials. Cardanol (Cardolite® NX-2022) was provided by Cardolite Corporation. Hexane (99.5%, Millipore-Sigma), ethyl acetate (EA, HPLC grade, Fisher Scientific), tetrahydrofuran (THF, HPLC grade, Fisher Scientific), meta-chloroperoxybenzoic acid (mCPBA, <77%, Millipore-Sigma), palladium on carbon (Pd/C, 10%, Millipore-Sigma), dichloromethane (DCM, anhydrous, Millipore-Sigma), and sodium bicarbonate (NaHCO₃, Millipore-Sigma) were purchased and used without purification. DL-lactide was purchased from Corbion and recrystallized from toluene two times. 1,4-Benzenedimethanol (BDM, Millipore-Sigma) was dried over 72 h at a vacuum prior to use. Toluene was passed through a solvent drying system containing columns of activated alumina and molecular sieves. Sn(Oct)₂ (Millipore-Sigma) was distilled three times under vacuum with argon (30-50 mTorr, 130-150° C.) before storing under an inert atmosphere. The rosin ester tackifier (Sylvalite 2E 80HP) was provided by Arizona Chemical. Poly(ethylene terephthate) (PETE) film, a FINAT rubber hand roller (˜2 kg), stainless steel panels (PSTC, 304 BRT, 18 gauge) were purchased from ChemInstruments. All other chemicals were used as received from Millipore-Sigma unless otherwise specified.

Synthesis of pentadecyl cyclohexanone (PDCHN). Cardanol (60 g), Pd/C (15 g), and hexane (330 mL) were charged into a 1 L high pressure reactor (Series 4520 Bench Top Reactors, 1L, Parr Instrument Company). The reactor was sealed and pressurized to 100 psig with hydrogen, and then vented to 30 psig after 5 min. This procedure was repeated two more times, and then the reactor was pressurized to 600 psig and heated to 80° C. with stirring. When the pressure dropped to 450 psig, the reactor repressurized to 600 psig. After 24 h, the reactor cooled down to room temperature, vented, and disassembled. The orange solution was filtered through Celite, washed with hexane, and the organic solvent was removed in vacuum. The crude product, the mixture of pentadecyl cyclohexanone and pentadecyl cyclohexanol, was purified by a column chromatography to yield a white powder. (EA:hexane, 1:10 vol.). Yield: 16.3 g (27.1%). ¹H NMR (CDCl₃, 500 MHz): δ 0.84-0.91 (t, 3H), 1.20-1.42 (br, 28H), 1.54-1.58 (s, 1H), 1.59-1.69 (m, 1H), 1.70-1.81 (br, 1H), 1.85-1.93 (d, 1H), 1.95-2.08 (m, 2H), 2.21-2.30 (m, 1H), 2.30-2.38 (m, 1H), 2.39-2.46 (m, 1H) (ppm).

Synthesis of pentadecyl caprolactone (PDCL). mCPBA (13.9 g, 0.06 mmol) was dissolved in anhydrous DCM (140 mL). The phase separated water content was removed by a glass pipet, and the solution was further dried over magnesium sulfate, followed by filtration into a 500 mL flask. The solution was cooled down using a salt-ice bath, and the solution of PDCH (16.0 g, 0.052 mmol) in MC (160 mL) was slowly added. The salt-ice bath was removed, and the flask was left to react while slowly warming up to room temperature, accompanied with the precipitation of m-chlorobenzoic acid (mCBA). After 4 h, the flask was cooled down using a salt-ice bath, and filtered to remove precipitated mCBA. The solution was washed with a freshly prepared 10% aqueous sodium bisulfite (×3), saturated sodium bicarbonate, and brine, followed by drying over magnesium sulfate. The solution was filtered and residual organic solvents were removed in vacuum. Yield: 12.6 g (74.8%). ¹H NMR (CDCl₃, 500 MHz): δ 0.83-0.93 (t, 3H), 1.17-1.38 (br, 28H), 1.39-1.49 (m, 1H), 1.59-1.69 (m, 1H), 1.70-1.82 (br, 1H), 1.83-2.00 (m, 2H), 2.48-2.68 (m, 2H), 3.99-4.29 (m, 2H) (ppm). MS-ESI: Mass calculated for C21H40O2Na, m/z=347.2926; found, m/z=347.2927.

Synthesis of PPDCL. PDCL, BDM, and Sn(Oct)₂ were added to a pressure vessel under an argon atmosphere in a glove box. The vessel was sealed, taken out of the glove box, and placed in the oil bath at an elevated temperature. After a certain reaction time, the vessel cooled in an ice bath to quench the reaction. The polymer solution was precipitated into cold (−10° C.) methanol (×3). A white polymer powder was collected by a filtration and dried under vacuum for 48 h (yield: 80-93%). ¹H NMR (CDCl₃, 500 MHz): δ 0.82-0.93 (t, 785H), 1.08-1.47 (br, 7770H), 1.48-1.69 (m, 733H), 1.70-2.07 (br, 197), 2.19-2.32 (m, 470H), 3.92-4.08 (m, 456H), 5.09-5.12 (s, 4H) (ppm).

Synthesis of block copolymers. Poly(lactide)-block-poly(pentadecyl caprolactone)-block-poly(lactide) (LPL) was synthesized by the ring-opening transesterification polymerization of lactide using hydroxy telechelic PPDCL macro-initiator. Hydroxy telechelic PPDCL, DL-lactide, Sn(Oct)₂, and toluene were added to a pressure vessel under an argon atmosphere in a glove box, making a 30 wt % solution. The vessel was sealed, taken out of the glove box, and placed in a 110° C. oil bath. After a certain reaction time, the vessel cooled in an ice bath to quench the reaction. The polymer solution was precipitated into cold (−10° C.) methanol (×3). A white polymer powder was collected by a filtration and dried under vacuum for 48 h (yield: 80-93%). ¹H NMR of LPL(125 k, 0.06) (CDCl₃, 500 MHz): δ 0.84-0.92 (t, 997H), 1.09-1.42 (br, 10106H), 1.47-1.68 (m, 1699H), 1.82-1.92 (br, 131H), 2.20-2.33 (br, 654H), 3.92-4.09 (m, 640H), 5.10-5.11 (s, 4H), 5.12-5.27 (m, 140H) (ppm). The same procedures were used for the synthesis of poly(L-lactide)-block-poly(pentadecyl caprolactone)-block-poly(L-lactide) (cLPL) except for using L-lactide instead of using DL-lactide.

Adhesive testing. Standard test methods used for peel adhesion, loop tack, and shear strength were PSTC:101, ASTM:D1695, and PSTC:107, respectively 1-4 Polymer (20-100 mg) and tackifier (0-80 mg) were dissolved in THF, yielding a 30 wt % solution. Samples for adhesion test were prepared by casting the polymer solution onto a PETE film (50 μm) using a wire wound rod, followed by drying under ambient condition for 24 h. The thickness of the dried film was approximately 100 μm. The polished stainless steel panel (PSTC 304 BRT, 18 Gauge) was used as the adherends for adhesion testing.

Peel test. In a 180° peel test, a 1 cm wide strip of the PSA coated-PETE film was adhered to a stainless steel panel using a 2 kg rubber roller. The sample was tested by a Shimadzu ASG-X tensile tester at a peel rate of 305 mm min⁻¹. A slow peel rate of 31 mm min⁻¹ was applied when a stick slip behavior was observed. The peel force was recorded as the maximum measure force. The test was performed at least three times and averaged across the samples except for deviant samples.

Loop tack test. A 1 cm wide strip of the PSA coated-PETE film was made into a teardrop shaped loop and mounted to the upper grip of the tensile tester while a stanless steel panel was mounted on the lower grip. Then the loop was gently lowered forming a contact area of 1 cm×1.5-2 cm. The tack force was monitored while the upper grip was lifted at a rate of 305 mm min⁻¹. The tack force was recorded as the maximum measure force. The test was performed at least three times and averaged across the samples except for deviant samples.

Shear test. A PSA coated-PETE film was adhered to a stainless steel panel forming a contact area of 1.27 cm×1.27 cm, and pressed by a rubber roller. A 500 g weight was applied to the sample, and the time to failure (weight drop) was recorded, and averaged across at three samples.

Nuclear magnetic resonance (NMR). NMR spectroscopy data were obtained using a 500 MHz Bruker Avance III HD spectrometer with a SampleXpress autosampler (HD-500). All NMR data were analyzed by using the TopSpin (Bruker).

Fourier-transform infrared (FT-IR). FT-IR spectra of PDCL were measured using a Nicolet 6700 FT-IR spectrometer in ATR mode by a MCT-A detector (Thermo Fisher Scientific).

Differential scanning calorimetry (DSC). DSC were performed using a Mettler Toledo DSC 1 instrument. 5-10 mg of sample was loaded into hermetically sealed aluminum pans for a run. The thermal history of all the samples was removed by the first heating (10° C. min⁻¹) to 180° C. followed by the quenching to −200° C. The DSC thermograms are the second heating thermograms (10° C. min⁻¹).

Size exclusion chromatography (SEC). SEC was performed in THF (25° C., 1 mL min⁻¹) on an Agilent Infinity 1260 HPLC system equipped with three Waters Styragel HR columns, a Wyatt HELEOS-II multiangle laser light scattering (MALS) detector, and a Wyatt Optilab T-rEX differential refractive index detector.

Mass spectroscopy (MS). Molar mass of PDCL was determined by the Bruker BioTOF II ESI/TOF-MS. Approximately 1 mg of PDCL was dissolved in 10 mL of methanol, and diluted with methanol in a syringe just prior to a shot. The obtained molar mass was calibriated by poly(ethylene glycol) (Mn=200 g mol⁻¹).

Rheological properties. Rheological properties were measured with an ARES rheometer (TA Instruments) equipped with an 8 mm parallel-plate fixture. All experiments were carried out in the linear viscoelastic region, which was determined by dynamic strain sweeps. Temperature dependence of storage modulus (G′), loss modulus (G″), tan δ (G″/G′) were determined by dynamic temperature sweeps under a frequency of 1 rad s⁻¹ with a cooling rate of 10° C. min⁻¹. Further analysis below −65° C. was not accessible due to the maximum torque limit of the equipment.

Density measurement. PPDCL was melted and densified using a hot plate at 50° C. followed by a cooling to 0° C. (−20° C. min⁻¹). The density of PPDCL was measured at 0° C. (ice bath) by the Archimedes' principle with a density determination kit for Excellence XP/XS analytical balances (Mettler Toledo). The density measurements were repeated 7 times, and used the average value. The density of PPDCL was 0.943±0.014 g cm⁻³.

Synchroton small angle X-ray scattering. Approximately 20 mg of polymer samples were placed in a TZero aluminum DSC pans (TA Instruments), and sealed in an argon-filled glove box to prevent oxidative degradation of the samples during the experiments. Synchrotron X-ray scattering experiments were performed at DND-CAT Sector (beamline 5-ID-D) at the Advanced Photon Source (APS) located at Argonne National Laboratory. Beam energies and sample-to-detector distances of 17 keV and 8.5 m were used. A Linkam DSC600 stage was used to control the heating and cooling cycles during the experiments. Prior to the measurement, the sample was heated to 160° C. and then cooled to 40° C. with a cooling rate of −10° C. min⁻¹. Two-dimensional scattering patterns were collected on a Rayonix CCD area detector. The resulting isotropic scattering patterns were azimuthally integrated to give the scattered intensity as a function of the magnitude of the scattering wave vector q=4π sin(θ/2)/λ, where θ is the scattering angle and λ (=0.7293 Å) is the wavelength of the incident beam. All the X-ray scattering data are in original form without a background subtraction.

Calculation of entanglement molar mass (Me). Assuming that LPL is forming a spherical morphology and PLA domains act as a filler in a rubbery matrix, the plateau modulus of LPL (GN,LPL) is predicted by the Guth-Smallwood equation (eq. 2) as follows:

G_{N,LPL}=G_{N,PPDCL}(1+2.5Φ+14.1Φ²) (eq. 2)

where G_{N,PPDCL} is the plateau modulus of PPDCL and Φ is the volume fraction of PLA blocks. After obtaining G_{N,PPDCL}, Me of PPDCL was calculated using eq. 3:

Me=ρRT/G_{N,PPDCL} (eq. 3)

where ρ is the density of PPDCL, R is universal gas constant, and T is the absolute temperature.

Degradation experiment. Polymers were dissolved in THF with a concentration of 8 mg mL⁻¹. Aqueous solution of 0.1 M HCl or NaOH was added yielding 4 mg mL⁻¹ polymer solution. The vials containing the solution were placed in the aluminum beads bath (50° C.). The aliquot was taken at the predetermined time, and the solvent was evaporated in the vacuum oven ovenight. The molar mass of the remained solid was measured by MALS-SEC (THF, 25° C.).

### Copolymerization of Pentadecyl Caprolactone and Methyl Caprolactone

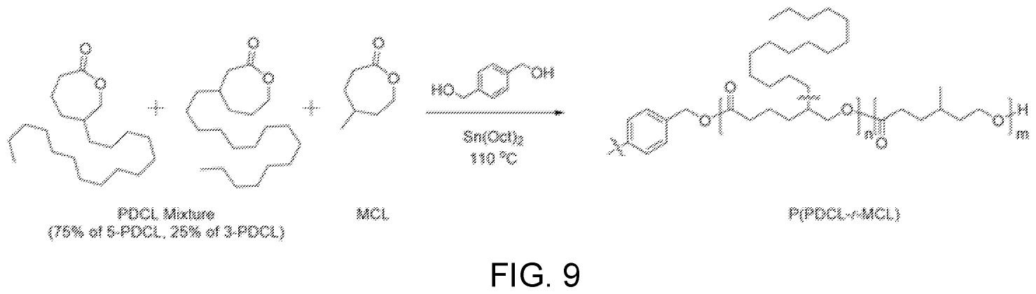

Copolymerization of PDCL and MCL was performed at 110° C., [M]o/[I]o=250, [PDCL]₀:[MCL]₀=65:35, [Sn(Oct)₂]₀=0.007 M, [M]₀=1 M in toluene (FIG. 9). ¹H NMR spectrum of a random copolymer of PDCL and MCL, P(PDCL-r-MCL), shows characteristic chemical shifts of both PDCL and MCL units. For example, chemical shifts for the methyl-end (—CH₃) of PDCL and MCL were observed at δ=0.88 ppm (assigned as a) and δ=0.92 ppm (assigned as h), respectively. Based on the ¹H NMR spectrum, molar composition of [PDCL]:[MCL] in the polymer is 63:37. Unimodal SEC chromatogram (Mₙ=62.3 kDa, Ð=1.1) indicates that the copolymerization was performed in a controlled fashion. DSC data of P(PDCL-r-MCL) shows T_{g}=−35° C. and Tₘ=14° C. Decrease in Tₘ upon the copolymerization is attributed to the incorporation of amorphous MCL units, which prevent the crystallization of PDCL units.

Synthesis of copolymer. Pentadecyl caprolactone (PDCL), methyl caprolactone (MCL), benzene dimethanol (BDM), Sn(Oct)₂, and toluene were added to a pressure vessel under an argon atmosphere in a glove box. The vessel was sealed, taken out of the glove box, and placed in the oil bath at 110° C. After a certain reaction time, the vessel cooled in an ice bath to quench the reaction. The polymer solution was precipitated into cold (−10° C.) methanol (×3). A viscous polymer liquid was collected and dried under vacuum overnight (yield: 82%).

Nuclear magnetic resonance (NMR). NMR spectroscopy data was obtained using a 500 MHz Bruker Advance III HD with a SampleXpress autosampler (HD-500). NMR data was analyzed by using the TopSpin (Bruker).

Size exclusion chromatography (SEC). SEC was performed in THF (25° C., 1 mL min⁻¹) on an Agilent Infinity 1260 HPLC system equipped with three Waters Styragel HR columns, a Wyatt HELEOS-II multiangle laser light scattering (MALS) detector, and a Wyatt Optilab T-rEX differential refractive index detector.

Differential scanning calorimetry (DSC). DSC was performed using a TA Instruments Discovery DSC. Approximately 10 mg of sample was loaded into hermetically sealed aluminum pans for a run. The sample was heated at 20° C. min⁻¹ to 180° C. followed by cooling at −10° C. min⁻¹ to −100° C. Then, sample was reheated at 10° C. min⁻¹ to 200° C. to obtain glass transition temperature (T_{g}) and melting temperature (Tₘ).

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

1. A method of synthesizing a polymer, the method comprising:
polymerizing pentadecyl caprolactone to yield poly(pentadecyl caprolactone).

2. The method of claim 1, wherein polymerizing pentadecyl caprolactone comprises polymerizing pentadecyl caprolactone with one or more additional monomers to yield a copolymer comprising poly(pentadecyl caprolactone).

3-20. (canceled)

21. A polymer comprising poly(pentadecyl caprolactone).

22. The polymer of claim 21, wherein the polymer is a copolymer or a block copolymer.

23. (canceled)

24. The polymer of claim 22, wherein block copolymer comprises a first block type and a second block type, wherein the first block type and the second block type are different.

25. The polymer of claim 24, wherein the block copolymer further comprises a third block type.

26. The polymer of claim 25, wherein the third block type is different than the first block type and the second block type.

27. The polymer of claim 25, wherein the third block type is the same as the first block type or the second block type.

28. The polymer of claim 24, wherein the first block type comprises poly(lactic acid).

29. The polymer of claim 27, wherein the polymer is an ABA triblock copolymer.

30. The polymer of claim 29, wherein the ABA triblock copolymer is represented by the following chemical structure: 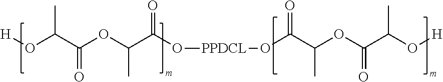
wherein each m is independently an integer, and PPDCL is represented by the following chemical structure:

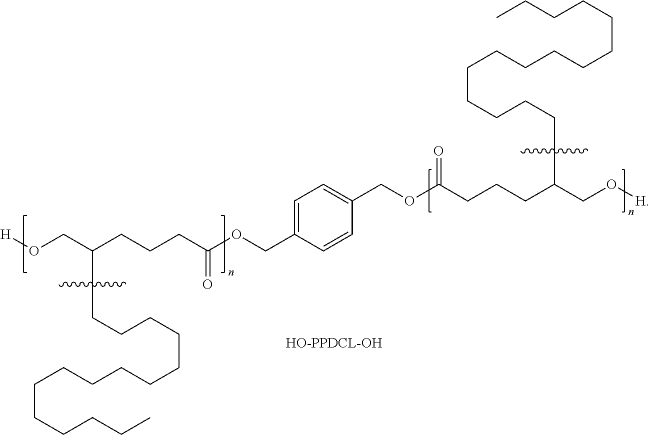

31. The polymer of claim 30, where the first block comprises, consists essentially of, or consists of poly(L-lactic acid), and the ABA triblock copolymer is represented by the following chemical structure:

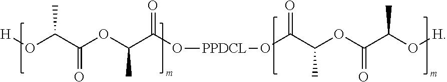

32. The polymer of claim 21, wherein the polymer degrades to carbon-containing molecules comprising 20 to 200 carbon atoms.

33. The polymer of claim 24, wherein the first block and the second block are immiscible.

34. The polymer of claim 24, wherein the first block comprises a polymer having a glass transition temperature exceeding that of PPDCL.

35. The polymer of claim 34, wherein the block copolymer is represented by one of the following chemical structures:

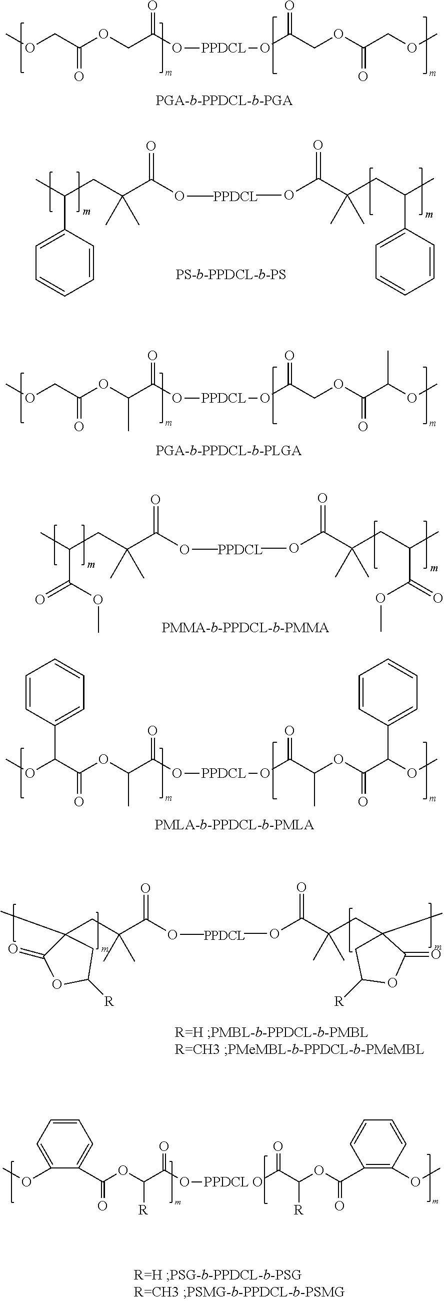

36. The polymer of claim 24, wherein the glass transition temperature of the first block is greater than 25° C.

37. The polymer of claim 24, wherein the first block comprises poly(glycolic acid) (PGA), poly(lactic acid-co-glycolic acid) (PLGA), poly(mandelic acid-co-lactic acid) (PMLA), poly(salicylic glycolid) (PSG), poly(salicylic methyl glycolide) (PSMG), poly(styrene) (PS), poly(methyl methacrylate) (PMMA), cpoly(α-methylene-γ-butyrolactone) (PMBL), or poly(γ-methyl-α-methylene-γ-butyrolactone) (PMeMBL).

38. A pressure sensitive adhesive comprising the polymer of claim 21.

39. The pressure sensitive adhesive of claim 38, further comprising a tackifier.

40-43. (canceled)
